(19) United States
(12) Patent Application Publication
(10) Pub. No.: US 2020/0315574 A1
(43) Pub. Date: Oct. 8, 2020

(54) APPARATUS AND INFORMATION PROCESSING METHOD

(71) Applicant: CANON KABUSHIKI KAISHA, Tokyo (JP)

(72) Inventors: Ryuichi NANAUMI, Tokyo (JP); Yukio FURUKAWA, Sagamihara-shi (JP); Kazuhiko FUKUTANI, Yokohama-shi (JP)

(73) Assignee: Canon Kabushiki Kaisha, Tokyo (JP)

(21) Appl. No.: 16/304,835

(22) PCT Filed: Jun. 16, 2016
(86) PCT No.: PCT/JP2017/023113

§ 371 Date: Nov. 27, 2018

(30) Foreign Application Priority Data
Jun. 24, 2016 (JP) 2016-125805

(51) Int. Cl. A61B 8/00; A61B 8/08; A61B 8/14

(52) U.S. Cl. CPC A61B 8/4281; A61B 8/406; A61B 8/523; A61B 8/08; A61B 8/14; A61B 8/58

(57) ABSTRACT

An apparatus that acquires specific information on a subject on the basis of a signal originating from an acoustic wave which has propagated from the subject and propagated through an acoustic medium disposed between the subject and a receiving unit is provided. The apparatus includes: a wave number acquiring unit acquiring a spatial wave number component of the signal; a correcting unit correcting the spatial wave number component using parameters of a longitudinal wave velocity in the subject and a transverse wave velocity in the acoustic medium; and an information acquiring unit acquiring the specific information using the corrected spatial wave number component.

### TECHNICAL FIELD

The present invention relates to an apparatus and an information processing method.

### BACKGROUND ART

Conventionally, a subject information acquiring apparatus such as a photoacoustic imaging apparatus and an ultrasound echo imaging apparatus has been proposed as a technique for receiving acoustic waves to acquire information on the inside of a subject such as a living body.

For example, it has been found that a photoacoustic imaging apparatus is useful for diagnosing skin cancer or breast cancer, in particular. There is an increasing expectation that the photoacoustic imaging apparatus will be used as a medical apparatus which replaces an ultrasound echo diagnosis apparatus, an X-ray apparatus, an MRI apparatus, and the like which have conventionally used for these diagnoses.

When a living body tissue is irradiated with a measurement light beam such as visible light or near-infrared light, a light absorbing substance inside the living body, such as hemoglobin in the blood, for example, absorbs the energy of the measurement light beam to expand instantaneously. As a result, an acoustic wave is generated. This phenomenon is referred to as a photoacoustic effect and the generated acoustic wave is referred to as photoacoustic wave.

The photoacoustic imaging apparatus visualizes the information on the living body tissue by measuring this photoacoustic wave. A tomography technique which uses such a photoacoustic effect is also referred to as photoacoustic imaging (PAI).

The photoacoustic imaging can image information related to an absorption coefficient of the inside of a subject. The absorption coefficient is the ratio of the optical energy absorbed by a living body tissue. An example of information related to the absorption coefficient is initial sound pressure which is the sound pressure at the instant at which a photoacoustic wave is generated. The initial sound pressure is proportional to the product between optical energy (light intensity) and the absorption coefficient. Therefore, it is possible to obtain the absorption coefficient by performing an appropriate process on the value of the initial sound pressure.

Furthermore, the absorption coefficient depends on the concentrations of components that constitute the living body tissue. Therefore, it is possible to acquire the concentrations of these components from the absorption coefficient. Particularly, by using light having a wavelength which is easily absorbed in hemoglobin in the blood, it is possible to acquire the concentration ratio between oxygenated hemoglobin and reduced hemoglobin and the oxygen saturation of the living body tissue. There are expectations of applications to medical diagnosis in such a way that an oxygen saturation distribution is analyzed to identify tumor tissues in a living body or surrounding tissues thereof.

Patent Literature 1 discloses an apparatus that acquires an ultrasound image by taking refraction of sound rays by a holding member located between a subject and a transducer into consideration.

Non Patent Literature 1 and 2 disclose an image reconstruction method in a spatial wave number region.

Patent Literature 2 discloses an apparatus that acquires an ultrasound image by taking modulation of a spatial wave number component by a holding member located between a subject and a transducer into consideration.

### CITATION LIST

### Patent Literature

PTL 1: US Patent No. 6607489

PTL 2: Japanese Patent Application Publication No. 2015-027445

### Non Patent Literature

NPL 1: “k-Wave: MATLAB toolbox for the simulation and reconstruction of photoacoustic wave fields”, Journal of Biomedical Optics 15 (2), 021314 (March/April 2010)

NPL 2: “A simple Fourier transform-based reconstruction formula for photoacoustic computed tomography with a circular or spherical measurement geometry”, Proc. of SPIE Vol. 8581 85814K-1

### SUMMARY OF INVENTION

### Technical Problem

When such a holding member included in the apparatus disclosed in Patent Literature 1 is present in an acoustic medium through which an acoustic wave propagates, since the holding member is a solid, a portion of the acoustic wave which is a longitudinal wave is transformed into a transverse wave. When a transverse wave propagates through the holding member to reach the transducer or reach an acoustic medium on a side where the transducer is present, the transverse wave is transformed again into a longitudinal wave. In this case, a sound ray of an acoustic wave refracted by the holding member travels along a path different from that of the longitudinal wave and the transverse wave. Furthermore, the velocities of the longitudinal wave and the transverse wave when propagating through the holding member are different.

In any of the methods of Patent Literature 1 and 2 in which refraction by the holding member is corrected at one velocity only, it was difficult to deal with the refraction of both the longitudinal wave and the transverse wave. Due to this, in these conventional methods, an acoustic wave of one of the longitudinal wave and the transverse wave—the one which is not dealt with—may be, for instance, defocused during imaging and the image quality may deteriorate.

In Patent Literature 1 and 2, it was difficult to correct modulation of an amplitude received when an acoustic wave including a transverse wave passes through the holding member. Amplitude transmittance characteristics when the acoustic wave including the transverse wave passes through the holding member depend on an angle of incidence on the holding member and a frequency. Due to this, Fourier transform-based frequency conversion is required to correct the amplitude of an acoustic wave including the transverse wave according to the method disclosed in Patent Literature 1, and a considerable amount of computation time is required. Patent Literature 2 does not disclose a method for correcting the amplitude of the acoustic wave including the transverse wave and the correction is difficult. Due to these reasons, the contrast may, for instance, deteriorate during imaging and the image quality may deteriorate.

The present invention has been made in view of the above-described problems. An object of the present invention is to acquire high-accuracy information in a short period when specific information is acquired on the basis of an acoustic wave that propagates from a subject.

### Solution to Problem

According to an aspect of the present invention, there is provided an apparatus that acquires specific information on a subject on the basis of a signal originating from an acoustic wave which has propagated from the subject and propagated through an acoustic medium disposed between the subject and a receiving unit,
the apparatus comprising:
a wave number acquiring unit configured to acquire a spatial wave number component of the signal;
a correcting unit configured to correct the spatial wave number component using parameters of a longitudinal wave velocity in the subject and a transverse wave velocity in the acoustic medium; and
an information acquiring unit configured to acquire the specific information using the corrected spatial wave number component.

According to another aspect of the present invention, there is provided an apparatus that acquires specific information on a subject on the basis of a signal originating from an acoustic wave which has propagated from the subject and propagated through an acoustic medium disposed between the subject and a receiving unit,
the apparatus comprising:
a wave number acquiring unit configured to acquire a spatial wave number component of the signal;
a correcting unit configured to correct the spatial wave number component by deconvolution which uses information on a component based on a transverse wave in which a portion of the acoustic wave which is a longitudinal wave generated by the subject is transformed inside the acoustic medium; and
an information acquiring unit configured to acquire the specific information using the corrected spatial wave number component.

According to another aspect of the present invention, there is provided an information processing method of acquiring specific information on a subject on the basis of a signal originating from an acoustic wave which has propagated from the subject and propagated through an acoustic medium disposed between the subject and a receiving unit,
the method comprising:
a wave number acquisition step of acquiring a spatial wave number component of the signal;
a correction step of correcting the spatial wave number component using parameters of a longitudinal wave velocity in the subject and a transverse wave velocity in the acoustic medium; and
an information acquisition step of acquiring the specific information using the corrected spatial wave number component.

### Advantageous Effects of Invention

According to the present invention, it is possible to acquire high-accuracy information in a short period when specific information is acquired on the basis of an acoustic wave that propagates from a subject.

Further features of the present invention will become apparent from the following description of exemplary embodiments with reference to the attached drawings.

### BRIEF DESCRIPTION OF DRAWINGS

FIGS. 1A and 1B are sequence diagrams of a subject information acquiring apparatus and the periphery of a processing unit according to a first embodiment.

FIGS. 2A to 2D are diagrams illustrating the details of a probe according to the first embodiment.

FIGS. 3A to 3C are schematic diagrams of the subject information acquiring apparatus according to the first embodiment in which a holding member is replaceable.

FIG. 4 is a flowchart of a subject information acquiring method according to the first embodiment.

FIGS. 5A to 5C are schematic diagrams illustrating correction of a spatial wave number component.

FIG. 6 is a diagram illustrating a display unit.

FIGS. 7A and 7B are schematic diagrams of a subject information acquiring apparatus and the periphery of a processing unit according to a second embodiment.

FIG. 8 is a flowchart of a subject information acquiring method according to the second embodiment.

FIGS. 9A and 9B are diagrams illustrating a configuration of a subject information acquiring apparatus and the periphery of a processing unit according to a third embodiment.

FIG. 10 is a flowchart of a subject information acquiring method according to the third embodiment.

### DESCRIPTION OF EMBODIMENTS

Hereinafter, preferred embodiments of the present invention will be described with reference to the drawings. Dimensions, materials, shapes, relative arrangements, and the like of constituent components described below are to be appropriately changed according to the configuration and various conditions of an apparatus to which the present invention is applied. Therefore, the scope of the present invention is not limited to those described below.

The present invention relates to a technique of detecting an acoustic wave that is generated from a subject and propagates through an acoustic medium and generating and acquiring specific information on the inside of the subject. Therefore, the present invention may be understood as a subject information acquiring apparatus or a control method thereof, and alternatively, as a subject information acquiring method and a signal processing method. Moreover, the present invention may be understood as a program for allowing an information processing apparatus having a hardware resource such as a CPU or a memory to execute these methods, a computer-readable non-transitory storage medium having the program stored therein, and the information processing apparatus itself.

The subject information acquiring apparatus according to the present invention includes an apparatus which uses a photoacoustic tomography technique of irradiating a subject with light (electromagnetic waves) to receive an acoustic wave generates inside the subject or at a specific position on the surface of the subject according to a photoacoustic effect to acquire the specific information on the subject as image data. In this case, the specific information is information on the characteristic values corresponding to a plurality of positions inside the subject, generated using reception signals obtained by receiving a photoacoustic wave. Such a subject information acquiring apparatus may be referred to as a photoacoustic imaging in that the specific information on the inside of the subject is obtained in a format such as image data on the basis of photoacoustic measurement.

The specific information (photoacoustic specific information) originating from an electrical signal (photoacoustic signal) acquired by photoacoustic measurement is a value that reflects the rate of absorption of optical energy. For example, the specific information includes a generation source of the acoustic wave generated by light irradiation, an initial sound pressure inside the subject, or an optical energy absorption density or absorption coefficient derived from the initial sound pressure, a concentration of a substance that constitutes a tissue. Moreover, it is possible to calculate a blood component distribution such as an oxygen saturation distribution by obtaining an oxygenated hemoglobin concentration and a deoxygenated hemoglobin concentration as the substance concentration. Moreover, a glucose concentration, a collagen concentration, a melanin concentration, a volume fraction of fats and water, and the like are also obtained.

The subject information acquiring apparatus according to the present invention includes an apparatus which uses an echo technique of irradiating a subject with an acoustic wave to receive (detect) an acoustic wave having scattered and propagated after being reflected at a specific position inside the subject. Such a subject information acquiring apparatus may be referred to as an echo imaging apparatus and an ultrasound echo apparatus in that the specific information on the inside of the subject is obtained in a format such as image data on the basis of reflection and scattering characteristics of the acoustic wave. The specific information obtained by the echo imaging apparatus indicates an acoustic impedance difference in the subject and a position, a velocity, and a density at which the acoustic impedance is different.

A two-dimensional or three-dimensional specific information distribution is obtained on the basis of the specific information at respective positions of the subject. The distribution data may be generated as image data. The specific information may be obtained as distribution information at respective positions inside the subject rather than numerical data. That is, the specific information is distribution information such as an initial sound pressure distribution, an energy absorption density distribution, an absorption coefficient distribution, or an oxygen saturation distribution. Moreover, an acoustic impedance distribution, blood flow distribution information, and the like may be generated. Therefore, the present invention may be understood as an acoustic imaging apparatus, a control method thereof, and a program in that information based on an acoustic wave is visualized.

The acoustic wave referred to in the present invention is typically an ultrasound wave and include an elastic wave called a sound wave and an acoustic wave. An electrical signal converted from an acoustic wave by a probe is also referred to as an acoustic signal. However, the expressions, ultrasound waves or acoustic waves used in the present specification do not limit the wavelength of these elastic waves. The acoustic wave generated by the photoacoustic effect is referred to as a photoacoustic wave or a light-induced ultrasound wave. The electrical signal originating from a photoacoustic wave is also referred to as a photoacoustic signal. Moreover, an electrical signal originating from an echo wave generated when a transmitted ultrasound wave is reflected from a subject is also referred to as an ultrasound echo signal.

### FIRST EMBODIMENT

Hereinafter, an embodiment of the present invention will be described in detail with reference to the drawings. In principle, the same constituent elements will be denoted by the same reference numerals, and redundant description thereof will be omitted.

### Configuration of Subject Information Acquiring Apparatus

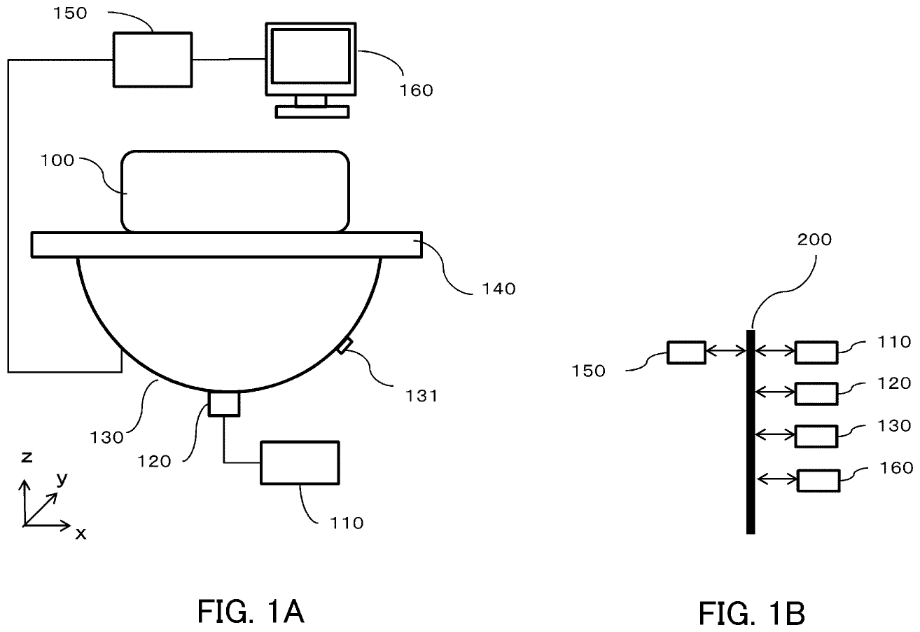

FIG. 1A is a schematic diagram of a subject information acquiring apparatus according to the present embodiment. Hereinafter, respective constituent elements of the apparatus will be described. The apparatus includes a probe 130 including a light source 110, an optical system 120, and a transducer 131, a holding member 140, a processing unit 150, and a display unit 160. A measurement target is a subject 100.

FIG. 1B is a schematic diagram illustrating the relation between the processing unit 150 and a peripheral configuration thereof. The processing unit 150 controls the operation of respective constituent elements of the subject information acquiring apparatus via a bus 200. Moreover, the processing unit 150 retains a program in which a subject information acquiring method to be described later is described and reads the program to cause the subject information acquiring apparatus to execute the subject information acquiring method. In the respective embodiments of the present invention, the processing unit 150 functions as a wave number acquiring unit, a correcting unit, and an information acquiring unit of the present invention. However, this does not necessarily mean that the processing unit 150 is physically divided into respective functions. For example, a program module that implements respective functions of a wave number acquiring process, a correction process, and an information acquiring process may operate in one information processing apparatus.

During photoacoustic measurement, first, light generated from the light source 110 is irradiated to the subject 100 held by the holding member 140 via the optical system 120. A photoacoustic wave is generated inside the subject 100 due to a photoacoustic effect. Subsequently, the probe 130 receives an acoustic wave having propagated through an acoustic medium to acquire a time-sequential electrical signal as a reception signal.

Hereinafter, the details of respective constituent elements of the subject information acquiring apparatus according to the present embodiment will be described. In the present embodiment, the technique of the present invention is applied to a photoacoustic apparatus. However, the present invention may be applied to an ultrasound echo apparatus. In this case, the subject information is information based on an echo wave of an ultrasound wave transmitted from the transducer 131 or an ultrasound wave transmission device rather than information based on a photoacoustic wave generated by a light source or an optical system.

### Subject 100

The subject 100 does not form a portion of the subject information acquiring apparatus of the present invention but the subject 100 will be described below. A major object of the subject information acquiring apparatus of the present invention is to examine blood diseases or malignant tumors of a person or an animal and to observe the progress of chemical treatments. Therefore, an examination target segment such as the breast, the neck, and the abdomen of a living body (specifically, a person or an animal) may be used as the subject.

A light absorber inside the subject has a relatively high absorption coefficient inside the subject. For example, when a human body is a measurement target, a blood vessel that contains a large amount of oxyhemoglobin or deoxyhemoglobin or a malignant tumor that contains many new-born blood vessels is a light absorber which is a measurement target. Besides this, plaque and the like on the carotid wall may be the measurement target.

### Light Source 110

A pulsed light source capable of generating a pulsed beam having a width of several nanoseconds to several microseconds is preferably used as the light source 110. Specifically, in order to generate a photoacoustic wave efficiently, the light source 110 is preferably capable of generating light having a pulse width of approximately 10 nanoseconds. A wavelength at which light propagates up to the inside of a subject is preferably used as the wavelength of light generated by the light source 110. Specifically, when the subject is a living body, a preferred wavelength is between 500 nm and 1200 nm. However, when an optical characteristic value distribution of a living body tissue relatively near the surface of a living body is obtained, a wavelength region (for example, between 400 nm and 1600 nm) wider than the wavelength region may be used.

A laser or a light emitting diode may be used as the light source. Various lasers such as a solid laser, a gas laser, a dye laser, or a semiconductor laser may be used as the laser. For example, in the present embodiment, an alexandrite laser, a Yttrium-Aluminium-Garnet laser, a Titan-Sapphire laser, and the like may be used.

Although a light source that generates light is used in this example, means for generating an electromagnetic wave may be used. For example, by using a microwave source, it is possible to acquire subject information by the same principle as the photoacoustic imaging. Moreover, by using a wavelength-variable laser capable of emitting light of a plurality of wavelengths, it is possible to generate a specific information distribution indicating a substance concentration such as an oxygen saturation in the blood on the basis of an absorption coefficient spectrum at each wavelength of each substance.

### Optical System 120

Light emitted from the light source 110 is propagated by an optical component of the optical system 120 and shaped into a desired light distribution shape and is guided to the subject 100. The optical component includes a mirror that reflects light, a lens that condenses and expands light and changes the shape thereof, a prism that disperses, refracts, and reflects light, an optical fiber that propagates light, a diffuser that diffuses light, and the like. Beside this, an arbitrary optical component may be used as long as the optical component can irradiate the light emitted from the light source 110 to the subject in a desired shape.

The intensity of light emitted from the optical system 120 to the subject 100 may be set in advance and may be stored in a storage unit 152 of the processing unit 150. The light source 110 is driven by a light source control unit (not illustrated) so as to irradiate irradiation light with this intensity. Alternatively, an optical sensor may be provided in the light source 110 or the optical system 120, a portion of light emitted actually may be measured, and the intensity of the irradiation light may be obtained and stored in the storage unit 152.

When it is possible to irradiate the light itself emitted by the light source 110 to the subject as desired light, it is not necessary to use the optical system 120. The optical system 120 or the light source 110, or a combination thereof may be understood as an irradiation unit.

### Probe 130

The probe 130 includes a transducer 131 which is a device capable of detecting an acoustic wave and a housing that surrounds the transducer. The transducer 131 receives an acoustic wave and converts the acoustic wave to an electrical signal which is an analog signal. An arbitrary device capable of receiving a photoacoustic wave such as a device which uses a piezoelectric phenomenon, resonance of light, a change in electrostatic capacitance, or the like may be used as the transducer 131.

A frequency component that forms a photoacoustic wave is typically between 100 KHz and 100 MHz. Therefore, the transducer 131 is preferably capable of detecting these frequencies.

The probe 130 includes a plurality of transducers 131. Due to this, it is possible to acquire photoacoustic waves generated by one instance of irradiation of light at a plurality of positions. As a result, the amount of information to be used for imaging increases and the image quality is improved.

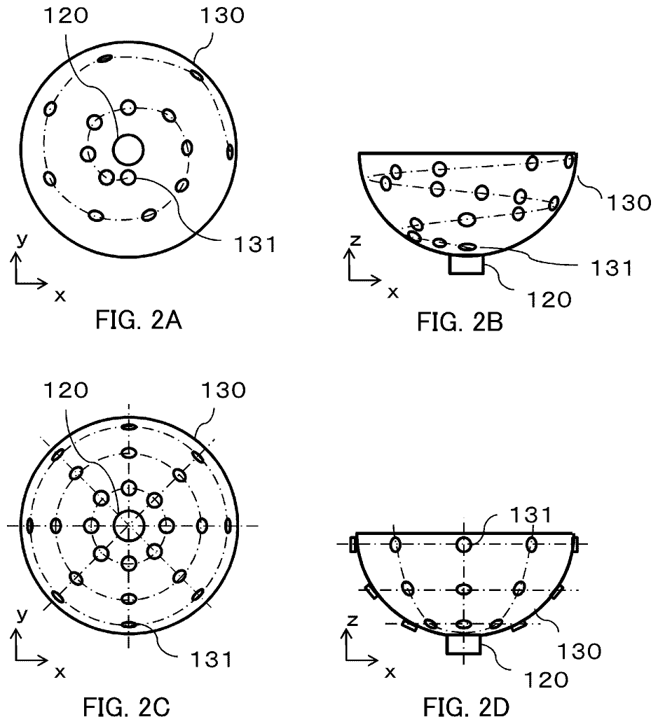

The probe 130 of the present embodiment has a spherical shape. FIG. 2 is a set of diagrams illustrating the details of the probe 130. FIGS. 2A and 2B illustrate the probe 130 in which the transducers 131 are arranged in a spiral form. FIGS. 2C and 2D illustrate the probe 130 in which the transducers 131 are arranged in a radial form. FIGS. 2A and 2C are diagrams of the probe 130 when seen from a z-axis direction of FIG. 1A, and FIGS. 2B and 2D are diagrams of the probe 130 when seen from a y-axis direction of FIG. 1A.

In any arrangement, since the transducers 131 are arranged on the spherical surface of the probe 130, it is possible to receive a photoacoustic wave generated by the subject 100 in various angular directions. Due to this, it is possible to generate specific information with high accuracy. In FIG. 2, although the transducers 131 are arranged in a spiral form or a radial form, an arrangement method is not limited thereto. For example, the transducers may be arranged on a spherical surface in a grid form.

The space between the probe 130 and the holding member 140 is filled with a matching member through which a photoacoustic wave can propagate. This matching member is preferably selected such that a photoacoustic wave can propagate through the acoustic medium, acoustic characteristics match at the interface between the holding member 140 and the transducer 131, and the transmittance of the photoacoustic wave is as high as possible. Furthermore, it is preferable that the matching member sufficiently transmit light emitted from the optical system 120. Water or oil, for example, can be used as the matching member. The probe 130 or the transducer 131, or both correspond to a receiving unit of the present invention.

### Holding Member 140

The holding member 140 has a function of mounting and holding the subject 100. By mounting the subject on the holding member, it is possible to suppress the motion during measurement, which may deteriorate the image quality. Furthermore, the holding member 140 has a function of pressing the subject by the weight and the like of the subject 100. By pressing and thinning the subject 100 so that light reaches up to the deep portion of the subject, the image quality at the deep portion of the subject 100 is improved. In order to increase these effects, the subject 100 may be sandwiched by a plurality of holding members 140. Moreover, by preventing the contact between the subject 100 and the probe 130 or the matching member, a health condition is improved and the safety is enhanced.

In order to attain the subject, an acoustic medium which has a certain degree of strength and is not easily deformed is preferably used as the holding member 140. Moreover, an acoustic medium which sufficiently transmits light emitted from the optical system 120 and an acoustic wave propagating from the subject 100 is preferred. For example, a resin material such as polycarbonate, polyethylene, or polyethylene terephthalate may be used. The holding member 140 corresponds to a holding unit of the present invention. However, the holding unit of the present invention may correspond to a combination of the holding member and the matching member rather than corresponding to the holding member only. Moreover, the holding member is not limited to a planar holding unit, but an arbitrary member capable of converting a propagated wave to a transverse wave corresponds to an acoustic medium of the present invention.

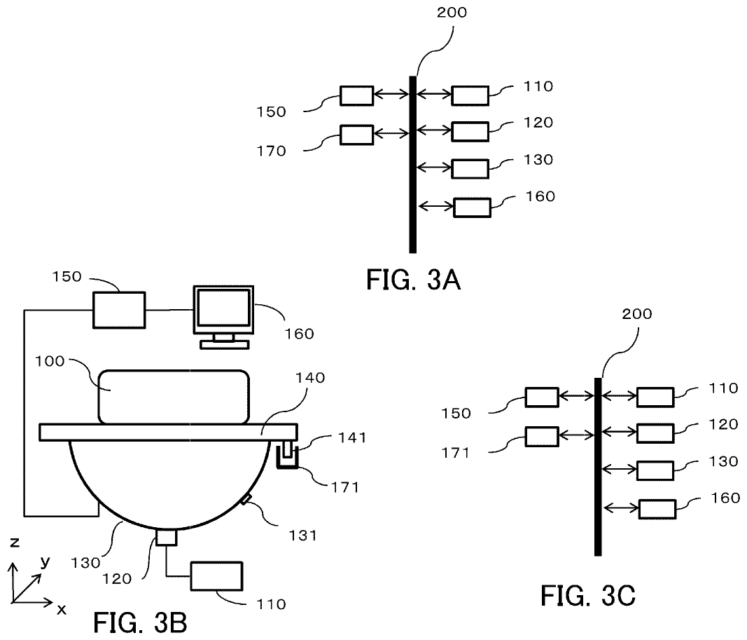

The holding member 140 may be replaceable. In this case, as illustrated in FIG. 3A, a holding unit specification acquiring unit 170 may be provided so that the specification of a replaced holding member can be acquired. According to an example of an acquisition method, the specification may be input by an operator. The input specification is set to the processing unit 150 via the bus 200. As the specification, the thickness of the holding member 140, a longitudinal wave velocity, a transverse wave velocity, and the like can be set.

More preferably, a tag 141 having specification information may be provided for each holding member. The specification information of the tag 141 is read by a reading unit 171 provided on a housing side of the apparatus and is set to the processing unit 150 via the bus 200 as illustrated in FIG. 3C. An acoustic matching member (water, oil, gel, or the like) is preferably disposed between the holding member 140 and the subject. Alternatively, a model number or the like of the holding member may be stored in the tag, and the specification acquiring unit may acquire the specification information stored in a memory on the basis of the model number. The specification corresponds to relating information of the present invention, and the specification acquiring unit corresponds to a relating information acquiring unit of the present invention.

A temperature sensor may be provided in the tag 141 to monitor the temperature of the holding member 140 so that the temperature-dependent specification values are corrected. In this way, it is possible to acquire specification information with high accuracy.

By setting the accurate specification of the holding member to the processing unit 150 in this manner, it is possible to execute a subject information acquiring method to be described later with higher accuracy.

### Processing Unit 150

The processing unit 150 performs an arithmetic operation for acquiring subject information on the inside of the subject using a reception signal. Typically, the processing unit 150 includes a device such as a CPU, a GPU, and an A/D converter and a circuit such as FPGA and ASIC. Moreover, the processing unit 150 may include a signal amplifier. Since the electrical signal converted from the acoustic wave by the transducer 131 is an analog signal, the electrical signal is generally converted to a digital signal and is amplified. The processing unit 150 may include a plurality of devices and a plurality of circuits rather than including one element and one circuit. Moreover, the respective processes performed by the subject information acquiring method may be executed by any of the devices and the circuits. Apparatuses that execute the respective processes are collectively referred to as a processing unit of the present invention.

The processing unit 150 preferably includes a storage unit 152 (not illustrated) having a memory function. Moreover, the processing unit 150 is preferably configured to process a plurality of signals simultaneously as pipeline processing. In this way, it is possible to shorten the time taken until subject information is acquired. The processing unit 150 has a non-transitory recording medium and can store respective processes performed by the subject information acquiring method as programs to be executed by the processing unit. A PC or the like having a processor, a storage means, and the like can be used as a portion of the processing unit 150 that implements an information processing function. In this case, a user interface (a keyboard, a mouse, and the like) of the PC can be used as the specification acquiring unit.

The processing unit 150 and the plurality of transducers 131 may be provided in a configuration in which these parts are included in a common housing. However, some signal processing may be performed by a processing unit included in the housing, and the remaining signal processing may be performed by a processing unit provided outside the housing. In this case, the processing units provided inside and outside the housing may be collectively referred to as a processing unit of the present invention. The processing unit 150 corresponds to a processor of the present invention.

### Display Unit 160

The display unit 160 is an apparatus that displays subject information output from the processing unit 150. Although a liquid crystal display or the like is typically used as the display unit 160, other displays such as a plasma display, an organic EL display, or FED may be used. The subject information may be displayed after image processing (adjustment of a brightness value and the like) is performed by the display unit 160 or the processing unit 150.

### Subject Information Acquiring Method

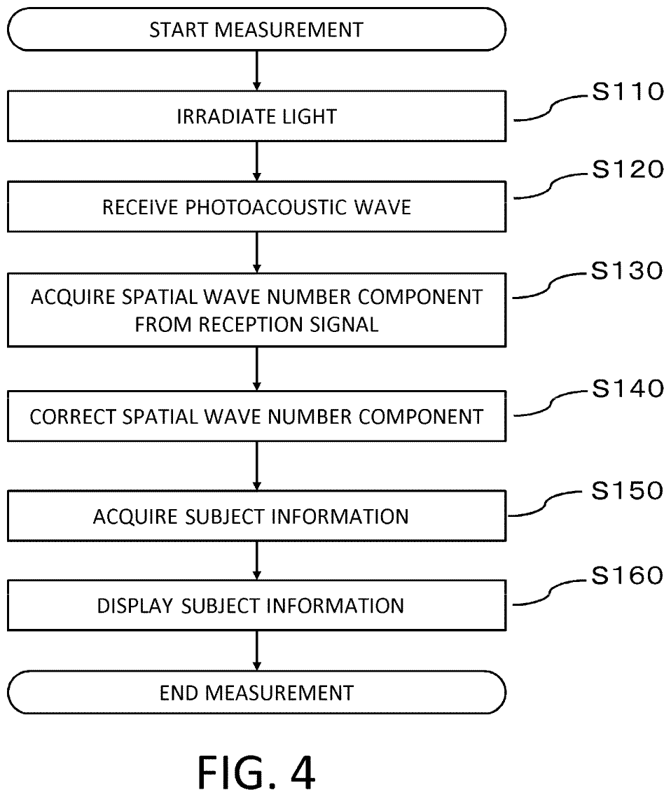

Next, respective steps of the subject information acquiring method according to the present invention will be described with reference to FIG. 4. The respective steps are executed when the processing unit 150 controls the operation of the respective constituent elements of the subject information acquiring apparatus.

### S110: Step of Irradiating Light to Inside of Subject to Generate Photoacoustic Wave

The light generated by the light source 110 is irradiated to the subject 100 as a pulsed beam via the optical system 120. The pulsed beam is absorbed inside the subject 100 and a photoacoustic wave is generated by a photoacoustic effect.

### S120: Step of Receiving Photoacoustic Wave to Acquire Reception Signal

In this step, the probe 130 receives (detects) a photoacoustic wave and outputs a reception signal from the transducer 131. The output reception signal is delivered to the processing unit 150. In this case, the reception signal (an analog electrical signal or an AD-converted digital electrical signal) may be stored in a memory of the processing unit 150 and information processing to be described later may be performed additionally. Alternatively, a corrected signal obtained in step S140 to be described later may be stored in a memory and image reconstruction may be performed additionally. Alternatively, a reception signal obtained from a certain region inside a region of interest may be processed while acquiring a photoacoustic wave from another region.

### S130: Step of Acquiring Reception Signal From Spatial Wave Number Component

In this step, a spatial wave number component of the reception signal is acquired. According to Non Patent Literature 2, a spherical probe can acquire a spatial wave number component p\hat{ } (kₓ, k_{y}, ω) of the reception signal according to Equation (1).

$$\begin{matrix}[ Math . 1 ] & \\ \hat{p} ( {k}_{x} , {k}_{y} , ω ) = \frac{2 {c}_{1}^{2}}{{R}^{s}} {\int }_{S} {dr}^{S} {e}^{ik \cdot {r}^{S}} Re \{ {F}_{1} \{ tp ( {r}^{S} , t ) \} ( {r}^{S} , ω ) \} & ( 1 )\end{matrix}$$

Here, kₓ indicates an x-component of a spatial wave number k, k_{y} indicates a y-component of the spatial wave number k, ω indicates a time-angular frequency, c₁ indicates a longitudinal wave velocity in the subject 100, R^{s} indicates the radius of curvature of the probe 130, S indicates the surface of the probe 130, r^{s} indicates a position vector of the transducer 131, and t indicates time. p(r^{s}, t) is the reception signal of the transducer 131 at the position of r^{s} and is a time-sequential signal. F₁{⋅} indicates a time Fourier transform and Re{⋅} indicates a real part.

Correctly, the spatial wave number component is represented by p\hat{ } (kₓ, k_{y}, k_{z}) using a z-component k_{z} of the spatial wave number k. However, since ω and k_{z} are coupled by the relation of Equation (2), p\hat{ } (kₓ, k_{y}, ω) is equivalent to the spatial wave number component.

[Math. 2]

ω=c₁|k|=c₁\sqrt{kₓ²+k_{y}²+k_{z}²} (2)

Since 2c₁²/R^{s} is a constant term, this is multiplied in S150 of acquiring subject information. Therefore, p\hat{ } (kₓ, k_{y}, ω) in Equation (3) is used as the spatial wave number component for the sake of convenience.

$$\begin{matrix}[ Math . 3 ] & \\ {\hat{p}}^{′} ( {k}_{x} , {k}_{y} , ω ) = {\int }_{S} {dr}^{S} {e}^{ik \cdot {r}^{S}} Re \{ {F}_{1} \{ tp ( {r}^{S} , t ) \} ( {r}^{S} , ω ) \} & ( 3 )\end{matrix}$$

Equation (3) means that the spatial wave number component can be acquired by performing a time Fourier transform on a multiplication result of time t and the reception signal p(r^{s}, t) of the transducer 131, taking the real part of the transform result, multiplying the real part with a weight and summing the multiplication result.

An arithmetic process may be performed on respective terms in the course of computation of Equation (3). For example, frequency filtering (low-pass filtering, high-pass filtering, band-pass filtering, and the like), deconvolution, envelope detection, wavelet filtering, and the like may be performed on F₁ {tp(r^{s}, t)} (r^{s}, ω). In this way, it is possible to improve the SN ratio or the like of the reception signal.

### S140: Step of Correcting Spatial Wave Number Component

In this step, amplitude and phase modulation including transform into transverse waves, received when an acoustic wave passes through the holding member 140 is corrected.

A complex amplitude transmittance h\hat{ } (kₓ, k_{y}, ω) of the acoustic wave set by taking interference due to reflection inside the holding member 140 into consideration is represented by Equation (4). That is, the acoustic wave is subjected to amplitude and phase modulation represented by Equation (4). Equation (4) can be derived by solving an equation by taking continuity of waves on a first surface and a second surface of the holding member 140 into consideration the waves being the longitudinal and transverse waves propagating in the reverse direction to the longitudinal and transverse wave propagating in a certain direction through the holding member 140.

$$\begin{matrix} [ Math . 4 ] & \\ \hat{h} ( {k}_{x} , {k}_{y} , ω ) = \frac{i 2 \frac{N cos {θ}_{1}}{{Z}_{3}}}{\frac{cos {θ}_{1} cos {θ}_{3}}{{Z}_{1} {Z}_{3}} ( {M}^{2} - {N}^{2} ) - 1 + iM ( \frac{cos {θ}_{1}}{{Z}_{1}} + \frac{cos {θ}_{3}}{{Z}_{3}} )} {e}^{( i \frac{ω T cos {θ}_{3}}{{c}_{3}} )} {e}^{( - i \vert k \vert T ( cos {θ}_{1} - \frac{{c}_{1}}{{c}_{3}} cos {θ}_{3} ) )} & ( 4 ) \\ N = F + G & ( 5 ) \\ M = F cos ( \frac{ω}{{c}_{2 L}} T cos {θ}_{2 L} ) + G cos ( \frac{ω}{{c}_{2 T}} T cos {θ}_{2 T} ) & ( 6 ) \\ F = \frac{{Z}_{2 L} {cos}^{2} 2 {θ}_{2 T}}{cos {θ}_{2 L} sin ( \frac{ω}{{c}_{2 L}} T cos {θ}_{2 L} )} & ( 7 ) \\ G = \frac{{Z}_{2 L} {sin}^{2} 2 {θ}_{2 T}}{cos {θ}_{2 L} sin ( \frac{ω}{{c}_{2 L}} T cos {θ}_{2 L} )} & ( 8 ) \\ {θ}_{1} = {θ}_{1} ( {k}_{x} , {k}_{y} , ω ) = {tan}^{- 1} \frac{\sqrt{{k}_{x}^{2} + {k}_{y}^{2}}}{\sqrt{{( \frac{ω}{{c}_{1}} )}^{2} - ( {k}_{x}^{2} + {k}_{y}^{2} )}} & ( 9 ) \\ \frac{sin {θ}_{1}}{{c}_{1}} = \frac{sin {θ}_{2 L}}{{c}_{2 L}} = \frac{sin {θ}_{2 T}}{{c}_{2 T}} = \frac{sin {θ}_{3}}{{c}_{3}} & ( 10 )\end{matrix}$$

c₁ is a longitudinal wave velocity in the subject 100, c_{2L} indicates a longitudinal wave velocity in the holding member 140, c_{2T} indicates a transverse wave velocity in the holding member 140, c₃ indicates a longitudinal wave velocity in a matching member in the probe 130, and T is a thickness of the holding member 140. Z₁ indicates a longitudinal wave acoustic impedance of the subject 100, Z_{2L} indicates a longitudinal wave acoustic impedance of the holding member 140, Z_{2T} indicates a transverse wave acoustic impedance of the holding member 140, and Z₃ indicates a longitudinal wave acoustic impedance of a matching member in the probe 130.

Equations (5) to (8) are functions introduced to simplify the notation of Equation (4). Equation (9) represents an angle of incidence θ₁ when a continuous planar wave of the spatial wave number component p\hat{ } (kₓ, k_{y}, ω) is incident on the holding member 140 from the subject 100. θ_{2L}, θ_{2T}, and θ₃ indicate a propagation angle of a longitudinal wave propagating inside the holding member 140, a propagation angle of a transverse wave propagating inside the holding member 140, and a propagation angle of a longitudinal wave propagating through a matching member inside the probe 130. As described above, the acoustic medium of the present invention includes a holding member (holding unit) and a matching member.

The cos terms included in Equations (4) to (8) can be computed using θ₁ obtained in Equation (9) and the Snell's law in Equation (10). Particularly, when c_{2L}>c₁, sinθ_{2L}>1 and cosθ_{2L} has an imaginary number. In this case, the sign is determined such that light attenuates while propagating in the direction vertical to the surface of the holding member 140. That is, Equation (11) is used.

[Math. 5]

cos θ_{2L}=−i\sqrt{sin²θ_{2L}−1} (11)

Equation (12) is used when cosθ_{2T} has an imaginary number.

[Math. 6]

cos θ_{2T}=−i\sqrt{sin²θ_{2T}−1} (12)

Deconvolution may be used in order to correct the spatial wave number component p\hat{ } (kₓ, k_{y}, ω) using h\hat{ } (kₓ, k_{y}, ω). For example, the Wiener filter of Equation (13) may be used. Convolution may be performed using a method other than the Wiener filter.

$$\begin{matrix}[ Math . 7 ] & \\ {\hat{p}}_{c} ( {k}_{x} , {k}_{y} , ω ) = \frac{conj ( \hat{h} ( {k}_{x} , {k}_{y} , ω ) )}{{\vert \hat{h} ( {k}_{x} , {k}_{y} , ω ) \vert}^{2} + {λ}^{2}} \cdot {\hat{p}}^{′} ( {k}_{x} , {k}_{y} , ω ) & ( 13 )\end{matrix}$$

The denominator λ is a parameter for suppressing amplification and zero-rate of noise and is generally set to approximately several to several tens percent of the maximum value of |h\hat{ }|.

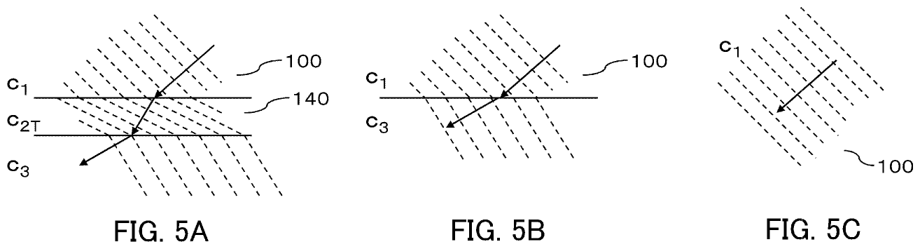

The second multiplication term exp(iωTcosθ₂/c₃) in Equation (4) has an effect of correcting a phase shift when the holding member 140 is replaced with a matching member. FIG. 5A illustrates a state in which a continuous planar wave of a certain spatial wave number component propagates through the subject 100, the holding member 140, and the matching member in that order. The planar wave propagates in the holding member at the transverse wave velocity c_{2T}. When the phase shift is corrected using the first to second multiplication terms of Equation (4), the spatial wave number component is corrected such that the wave propagates as illustrated in FIG. 5B.

The third multiplication term exp(−i|k|T(cosθ₁-c₁/c₃·cosθ₃)) has an effect of correcting a phase shift occurring due to a difference between the velocity c₁ in the subject and the velocity c₃ in the matching member. That is, a propagation path of the spatial wave number component illustrated in FIG. 5B is corrected to a propagation path illustrated in FIG. 5C. Since amplitude modulation at the interface between the velocities c₁ and c₃ is corrected by the first multiplication term of Equation (4), the third multiplication term may take correction of the phase shift only into consideration. Moreover, when c₁ and c₃ are sufficiently close values, the third multiplication term may be omitted from Equation (4) to simplify computation.

Equation (4) is an example of a complex amplitude transmittance which takes transverse wave transform into consideration. An arbitrary complex amplitude transmittance which takes transverse wave transform into consideration may be used for the deconvolution of Equation (13). For example, when the holding member 140 has a sufficient thickness and it is not necessary to take interference due to internal reflection into consideration, it is possible to use a complex amplitude transmittance which is set regardless of interference as illustrated in Equation (14).

$$\begin{matrix} [ Math . 8 ] & \\ {\hat{h}}^{′} ( {k}_{x} , {k}_{y} , ω ) = ( {t}_{12 L} {φ}_{2 L} ( ω ) {t}_{23 L} + {t}_{12 T} {φ}_{2 T} ( ω ) {t}_{23 T} ) {e}^{( i \frac{ω T cos {θ}_{3}}{{c}_{3}} )} {e}^{( - i \vert k \vert T ( cos {θ}_{1} - \frac{{c}_{1}}{{c}_{3}} cos {θ}_{3} ) )} & ( 14 ) \\ {t}_{12 L} = \frac{2 \frac{{Z}_{1}}{cos {θ}_{2 L}} cos 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{1}}{cos {θ}_{1}}} & ( 15 ) \\ {t}_{12 L} = \frac{- 2 \frac{{Z}_{1}}{cos {θ}_{2 T}} cos 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{1}}{cos {θ}_{1}}} & ( 16 ) \\ {φ}_{2 L} ( ω ) = exp ( - i \frac{ω T cos {θ}_{2 L}}{{c}_{2 L}} ) & ( 17 ) \\ {φ}_{2 T} ( ω ) = exp ( - i \frac{ω T cos {θ}_{2 T}}{{c}_{2 T}} ) & ( 18 ) \\ {t}_{23 L} = \frac{2 \frac{{Z}_{2 L}}{cos {θ}_{3}} cos 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{3}}{cos {θ}_{3}}} & ( 19 ) \\ {t}_{23 T} = \frac{- 2 \frac{{Z}_{2 T}}{cos {θ}_{3}} cos 2 {θ}_{2 T}}{\frac{{Z}_{2 L}}{cos {θ}_{2 L}} {cos}^{2} 2 {θ}_{2 T} + \frac{{Z}_{2 T}}{cos {θ}_{2 T}} {sin}^{2} 2 {θ}_{2 T} + \frac{{Z}_{3}}{cos {θ}_{3}}} & ( 20 )\end{matrix}$$

Equations (15) to (20) are functions introduced to simplify the notation of Equation (14). t_{12L} indicates a complex amplitude transmittance of a wave in which a continuous planar wave incident at the angle of incidence of θ₁ from the subject 100 to the holding member 140 passes through the holding member 140 as a longitudinal wave and t_{12T} indicates a complex amplitude transmittance of a wave in which the continuous planar wave passes through the holding member 140 as a transverse wave. Φ_{2L} and Φ_{2T} indicate the phase shifts that the longitudinal and transverse waves in the holding member 140 receive when propagating through the thickness T of the holding member 140, respectively. When cosθ_{2L} and cosθ_{2T} are imaginary numbers, these terms indicate attenuation. t_{23L} indicates a complex amplitude transmittance of a wave that passes from the holding member 140 toward the matching member in the probe 130 as a longitudinal wave and t_{23T} indicates a complex amplitude transmittance of a wave that passes as a transverse wave.

Equations (4) and (14) are examples calculated according to a boundary condition assuming that a physical amount at the boundary is continuous. A calculation result based on other boundary conditions such as a boundary condition which takes discontinuity of a physical amount at the boundary into consideration may be used.

A corrected spatial wave number component pc\hat{ } (kₓ, k_{y}, ω) corrected by Equation (13) in this manner is a spatial wave number component of a reception signal received when the matching member in the holding member 140 and the probe 130 is replaced with a matching member having the same acoustic characteristics (velocity) as the subject.

According to the present embodiment, it is possible to reduce a computation amount (time) by applying correction to amplitude modulation and phase modulation including transverse wave transform on the spatial wave number component acquired by image reconstruction in a spatial wave number region. In the case of a time-domain back-projection disclosed in Patent Literature 1, for example, since a transverse wave transform is dependent on an angle of incidence, the spectrum of a complex amplitude transmittance changes every voxel-transducer combination to be back-projected. That is, since a time Fourier transform is required for all voxel-transducer combinations, a computation amount of at least N³×N²×NlogN=N⁶logN is required (where N³ is the number of voxels, N² is the number of transducers, and N is the number of reception signal samples). More specifically, since N times of multiplication is required for ω excluding the variables kₓ and k_{y} in the deconvolution of Equation (13), the total computation amount required for correction is (N⁵ (NlogN+N)). On the other hand, in the present embodiment, since it is sufficient to execute Equation (13) on N³ spatial wave number components, the increase in the computation amount can be suppressed to N³. Furthermore, it is possible to halve the computation amount using the complex conjugate characteristics of the Fourier transform.

In the time-domain back-projection disclosed in Patent Literature 1, the deconvolution of Equation (13) is executed assuming that an acoustic wave radiates from a target voxel to be reconstructed. However, actually, a temporal signal included in the reception signal is an integrated value of all sound sources on a spherical surface from which the propagation time to the transducer is the same. Due to this, the angle of incidence θ₁ which is a parameter of the complex amplitude transmittance is sometimes not identical to the radiation direction of a signal, and the accuracy may decrease. On the other hand, in the present embodiment, a radiation direction of a certain spatial wave number component (or a continuous planar wave corresponding to the spatial wave number component) is determined uniquely from the spatial wave number by Equation (9). Due to this, Equation (13) can be executed with high accuracy.

The time-domain back-projection disclosed in Patent Literature 1 assumes that basically spherical waves are used. Due to this, this assumption does not coincide with the complex amplitude transmittance of Equation (4) which assumes the use of planar waves and the accuracy may decrease. In the present embodiment, since a spatial wave number component corresponding to a continuous planar wave is used, it is possible to strictly apply Equation (4) and execute the deconvolution of Equation (13) with high accuracy.

### S150: Step of Acquiring Subject Information

In this step, the processing unit 150 calculates an initial sound pressure distribution p₀(r) in the subject 100 using the corrected spatial wave number component acquired in step S140. When a region of interest includes a plurality of smallest imaging units (voxels or the like), an initial sound pressure of each smallest unit (that is, an initial sound pressure distribution in the region of interest) is calculated.

According to Non Patent Literature 2, it is possible to calculate the initial sound pressure distribution p₀(r) in the subject 100 from the spatial wave number component according to Equation (21).

$$\begin{matrix} [ Math . 9 ] & \\ {p}_{0} ( r ) = \frac{2 {c}_{1}^{2}}{{R}^{S}} {F}_{3}^{- 1} \{ {p}_{c} ( {k}_{x} , {k}_{y} , {k}_{z}^{′} ) \} = \frac{2 {c}_{1}^{2}}{{R}^{S}} {F}_{3}^{- 1} \{ {p}_{c} ( {k}_{x} , {k}_{y} , {ω}^{′} ) \} {\vert}_{{ω}^{′} = {c}_{1} \sqrt{{k}_{x}^{2} , {k}_{y}^{2} , {k}_{z}^{′2}}} \} & ( 21 )\end{matrix}$$

F₃⁻¹{⋅} indicates a three-dimensional inverse Fourier transform. k_{z}′ is a z-component of the spatial wave number component required when acquiring p₀(r). The discrete values ω and k_{z}′ obtained by the time Fourier transform of Equation (3) do not correspond to each other, the spatial wave number component of ω′ corresponding to k_{z}′ is interpolated from ω. Although an arbitrary interpolation method such as linear interpolation, sinc interpolation, or irregular interval interpolation may be used, it is preferable to use irregular interval interpolation having high accuracy. More preferably, fast irregular interval interpolation is used.

As described above, since the subject information is acquired using the corrected spatial wave number component p_{c}\hat{ } (kₓ, k_{y}, ω) obtained by correcting amplitude and phase modulation by the holding member 140, the defocusing or the like of the generated subject information is suppressed and the resolution, the contrast, and the like are improved.

### S160: Step of Displaying Subject Information

In this step, the subject information on the region of interest is displayed on the display unit 160 using the initial sound pressure distribution acquired in S150. An initial sound pressure distribution, an absorption coefficient distribution, an oxygen saturation, and the like may be displayed as the subject information. When the absorption coefficient distribution, the oxygen saturation, and the like are displayed, the processing unit 150 performs an arithmetic operation on the initial sound pressure distribution to acquire desired information.

Since the defocusing or the like of the subject information displayed on the display unit 160 is suppressed and the resolution and the contrast are improved, the subject information is suitable for use in diagnosis conducted by an operator such as a physician.

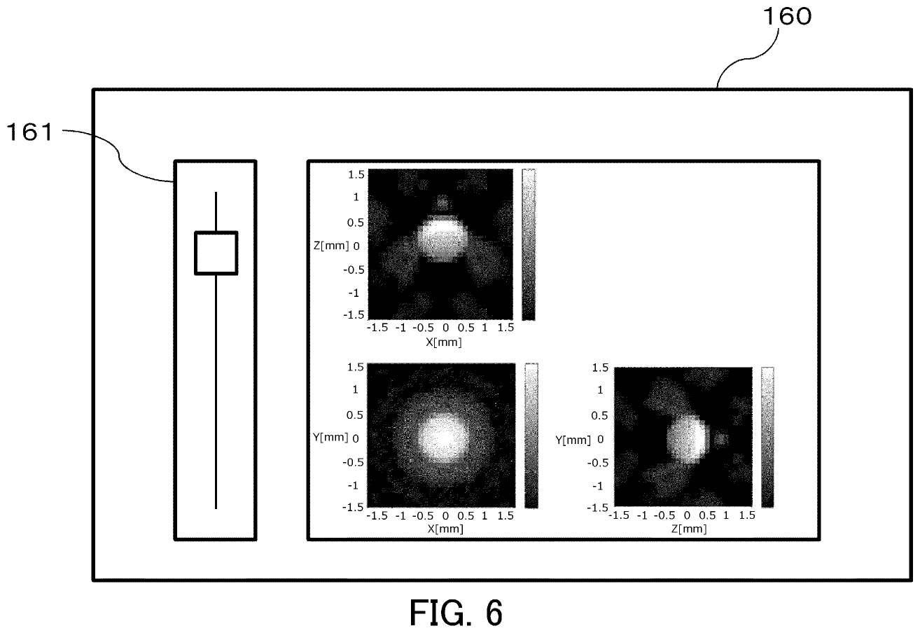

Means for changing the parameter λ in Equation (13) may be provided in the display unit 160. For example, a slider 161 illustrated in FIG. 6 may be provided and the parameter λ may be changed by an operator changing the position of the slider 161. Equations (13) and (21) are executed using the changed parameter λ, and the acquired subject information is displayed on the display unit 10. By doing so, it is possible to display information more suitable for use in diagnosis or the like conducted by an operator. The slider 161 may be a physical lever and may be an icon displayed on a GUI. The specification acquiring unit may also serve as the slider 161. The slider 161 corresponds to a correction characteristics adjusting unit of the present invention. Moreover, information corresponding to the individual characteristics of an examinee may be input by the function of the correction characteristics adjusting unit. For example, an age, a mammary gland density, past measurement information, and the like may be input.

According to the subject information acquiring method according to the present embodiment, it is possible to acquire subject information with high accuracy (image quality) in which defocusing or the like is suppressed and the resolution and the contrast are improved.

### SECOND EMBODIMENT

In the present embodiment, a subject information acquiring apparatus having a planar probe will be described. In principle, the same constituent elements as those of the first embodiment will be denoted by the same reference numerals, and redundant description thereof will be omitted.

### Configuration of Subject Information Acquiring Apparatus

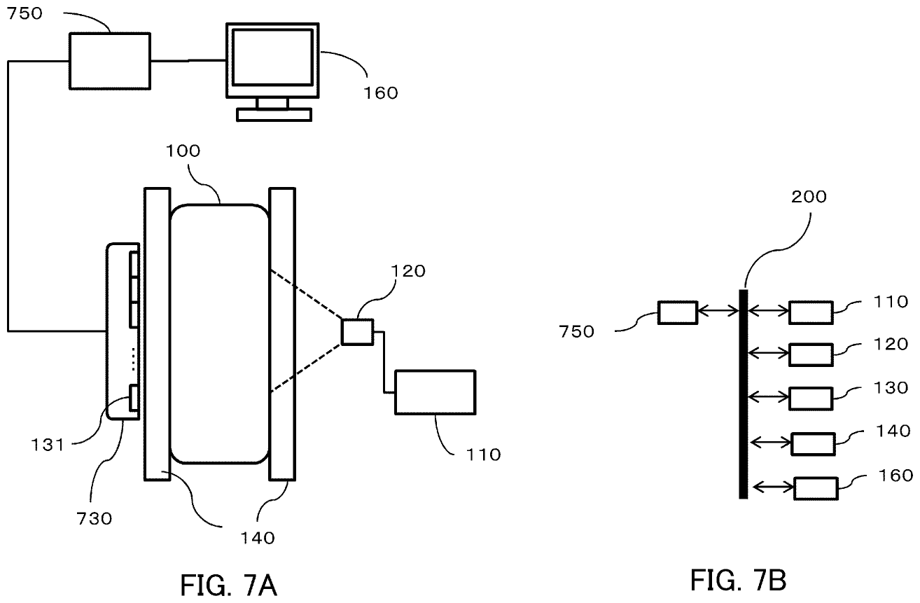

FIG. 7A is a schematic diagram of a subject information acquiring apparatus according to the present embodiment. The spherical probe 130 of the first embodiment is replaced with a planar probe 730. The probe 730 corresponds to a receiving unit of the present invention. FIG. 7B is a schematic diagram illustrating a relation between a processing unit 750 and a peripheral configuration thereof. The processing unit 150 of the first embodiment is replaced with a processing unit 750. The processing unit 750 corresponds to a processor of the present invention.

### Probe 730

The probe 730 includes a transducer 131 which is a device capable of detecting an acoustic wave and a housing that surrounds the transducer. In the present embodiment, the probe has a planar shape. The transducers 131 may be arranged in any arrangement as long as the transducers are arranged on a flat surface. For example, a grid arrangement, a sparse arrangement, a random arrangement, and the like may be employed. In order to shorten the computation time using a fast Fourier transform in a subject information acquiring method to be described later, the transducers are preferably arranged in a grid form.

The space between the probe 730 and the holding member 140 is filled with a matching member through which a photoacoustic wave can propagate. This matching member is preferably selected such that a photoacoustic wave can propagate through the acoustic medium, acoustic characteristics match at the interface between the holding member 140 and the transducer 131, and the transmittance of the photoacoustic wave is as high as possible. For example, water, oil, gel, or the like can be used.

### Subject Information Acquiring Method

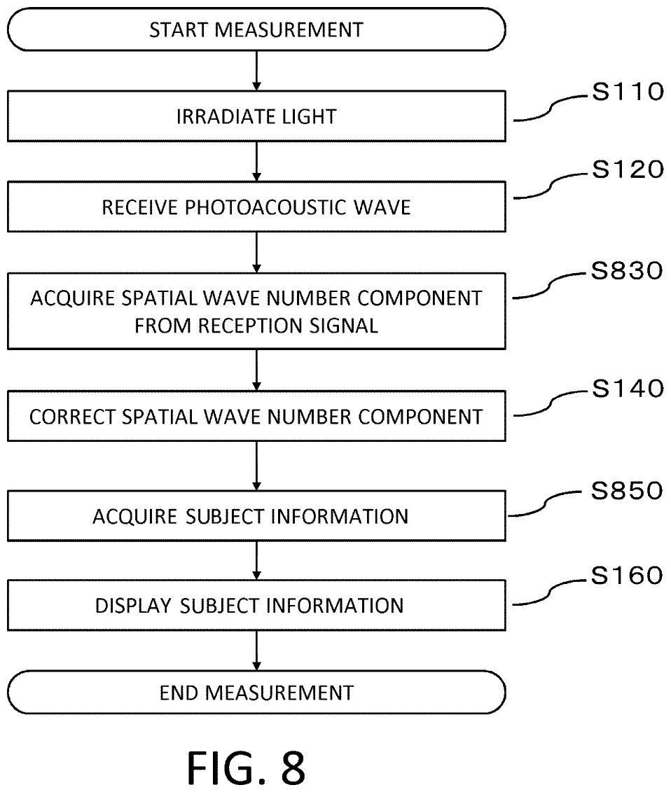

Next, respective steps of the subject information acquiring method according to the present embodiment will be described with reference to FIG. 8. The respective steps are executed when the processing unit 750 controls the operation of the respective constituent elements of the subject information acquiring apparatus.

S110, S120, S140, and S160 are the same as the steps having the same reference numerals of the first embodiment, and the description thereof will be omitted.

### 830: Step of Acquiring Spatial Wave Number Component From Reception Signal

In this step, the spatial wave number component of a reception signal is acquired. According to Non Patent Literature 1, a planar probe can acquire a spatial wave number component p\hat{ } (kₓ, k_{y}, ω) of the reception signal according to Equation (22).

$$\begin{matrix}[ Math . 10 ] & \\ \hat{p} ( {k}_{x} , {k}_{y} , ω ) = \frac{{c}_{1}^{2} \sqrt{{( \frac{ω}{{c}_{1}} )}^{2} - ( {k}_{x}^{2} + {k}_{y}^{2} )}}{2 ω} {F}_{x , y , t} \{ p ( x , y , t ) \} & ( 22 )\end{matrix}$$

Here, p(x, y, t) indicates a time-sequential reception signal of the transducer 131 on the basis coordinates x and y. F_{x, y, t}{⋅} indicates execution of a spatial Fourier transform on coordinates x and y and a time Fourier transform on time t.

Equation (22) has the following meaning. That is, a time Fourier transform is performed on the reception signals of the respective transducers 131 to acquire time and frequency components. Subsequently, a spatial Fourier transform is performed on the flat surface of the probe 730 for respective time and frequency components, whereby the spatial wave number component p\hat{ } (kₓ, k_{y}, ω) can be acquired.

An arithmetic process may be performed on respective terms in the course of computation of Equation (22). For example, frequency filtering (low-pass filtering, high-pass filtering, band-pass filtering, and the like), deconvolution, envelope detection, wavelet filtering, and the like may be performed on the result of the time Fourier transform. In this way, it is possible to improve the SN ratio or the like of the reception signal.

### S850: Step of Acquiring Subject Information

In this step, the processing unit 150 calculates an initial sound pressure distribution p₀(r) in the subject 100 using the corrected spatial wave number component acquired in step S140. When a region of interest includes a plurality of smallest imaging units (voxels or the like), an initial sound pressure of each smallest unit (that is, an initial sound pressure distribution in the region of interest) is calculated.

According to Non Patent Literature 1, it is possible to calculate the initial sound pressure distribution p₀(r) in the subject 100 from the spatial wave number component according to Equation (23).

$$\begin{matrix} [ Math . 11 ] & \\ {p}_{0} ( r ) = {F}_{3}^{- 1} \{ {p}_{c} ( {k}_{x} , {k}_{y} , {k}_{z}^{′} ) \} = {F}_{3}^{- 1} \{ {p}_{c} ( {k}_{x} , {k}_{y} , {ω}^{′} ) \} {\vert}_{{ω}^{′} = {c}_{1} \sqrt{{k}_{x}^{2} , {k}_{y}^{2} , {k}_{z}^{′2}}} \} & ( 23 )\end{matrix}$$

F₃⁻¹{⋅} indicates a three-dimensional inverse Fourier transform. k_{z}′ is a z-component of the spatial wave number component required when acquiring p₀(r). The discrete value ω obtained by the time Fourier transform of Equation (22) and kz′ do not correspond to each other, hence the spatial wave number component of ω′ corresponding to k_{z}′ is interpolated from ω. Although an arbitrary interpolation method such as linear interpolation, sinc interpolation, or irregular interval interpolation may be used, it is preferable to use irregular interval interpolation having high accuracy. More preferably, fast irregular interval interpolation is used.

As described above, since the subject information is acquired using the corrected spatial wave number component pc\hat{ } (kₓ, k_{y}, ω) obtained by correcting amplitude and phase modulation by the holding member 140, the defocusing or the like is suppressed and the resolution, the contrast, and the like are improved.

According to the subject information acquiring method according to the present embodiment, it is possible to acquire subject information with high accuracy (image quality) in which defocusing or the like is suppressed and the resolution and the contrast are improved using a planar probe.

### THIRD EMBODIMENT

In the present embodiment, a subject information acquiring apparatus capable of correcting phase modulation including a transverse wave transform with a small computation amount. Particularly, the present embodiment is suitable when the holding member 140 has a sufficient thickness. In principle, the same constituent elements as those of the first and second embodiments will be denoted by the same reference numerals, and redundant description thereof will be omitted.

### Configuration of Subject Information Acquiring Apparatus

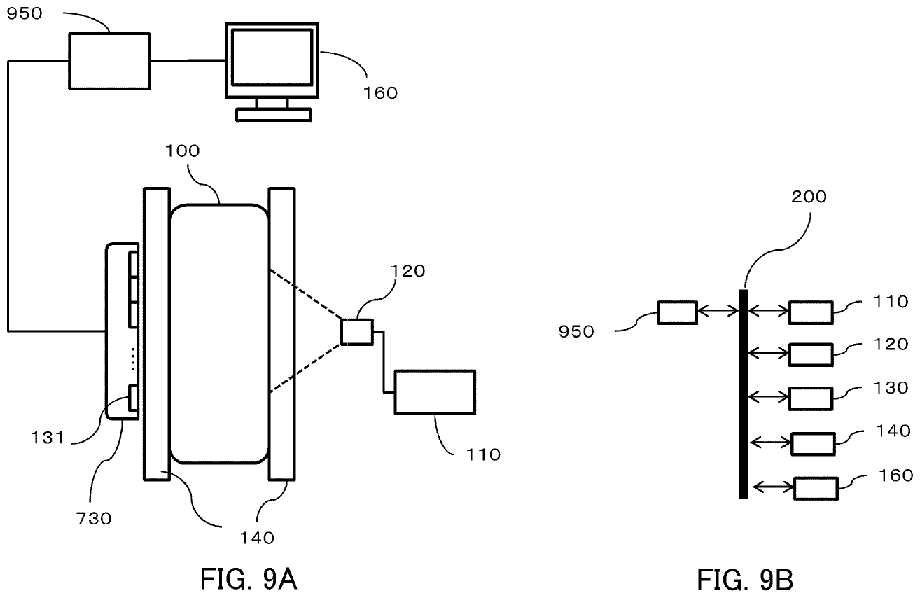

FIGS. 9A and 9B are a schematic diagram of a subject information acquiring apparatus according to the present embodiment and a schematic diagram illustrating the relation between a processing unit 950 and a peripheral configuration thereof. The processing unit 750 of the second embodiment is replaced with the processing unit 950. The processing unit 950 corresponds to a processor of the present invention.

### Subject Information Acquiring Method

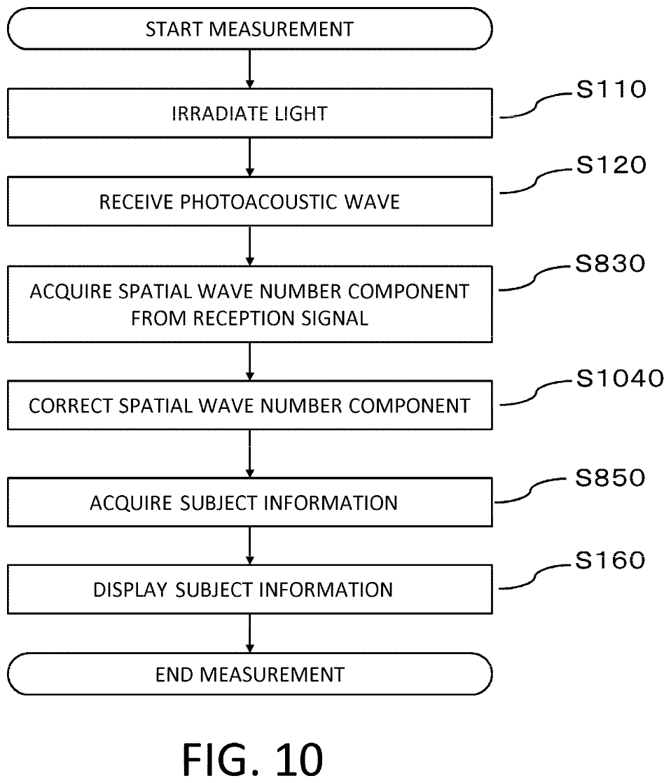

Next, respective steps of the subject information acquiring method according to the present embodiment will be described with reference to FIG. 10. The respective steps are executed when the processing unit 950 controls the operation of the respective constituent elements of the subject information acquiring apparatus.

S110, S120, S830, S850, and S160 are the same as the steps having the same reference numerals of the first or second embodiment, and the description thereof will be omitted.

### S1040: Step of Acquiring Reception Signal From Spatial Wave Number Component

In this step, phase modulation including transform to transverse waves, received when an acoustic wave passes through the holding member 140 is corrected.

According to Patent Literature 2, the phase modulation by the holding member 140 can be corrected by Equation (24).

$$\begin{matrix}[ Math . 12 ] & \\ {\hat{p}}_{c} ( {k}_{x} , {k}_{y} , ω ) = \hat{p} ( {k}_{x} , {k}_{y} , ω ) \cdot exp ( i \vert k \vert T ( cos {θ}_{1} - \frac{{c}_{1}}{{c}_{2}} cos {θ}_{2} ) ) & ( 24 )\end{matrix}$$

In the present embodiment (the present invention), correction is performed using both the longitudinal wave velocity c_{2L} and the transverse wave velocity c_{2T} as the velocity c₂ in the holding member 140. That is, Equation (25) which is a modification of Equation (24) is used.

$$\begin{matrix}[ Math . 13 ] & \\ {\hat{p}}_{c} ( {k}_{x} , {k}_{y} , ω ) = \hat{p} ( {k}_{x} , {k}_{y} , ω ) \cdot exp ( i \vert k \vert T ( cos {θ}_{1} - \frac{{c}_{1}}{{c}_{2}} cos {θ}_{2} ) ) \\ {c}_{2} = \{ \begin{matrix}{c}_{2 L} , & {θ}_{1} < {θ}_{c} \\ {c}_{2 T} , & {θ}_{1} \geq {θ}_{c}\end{matrix} & ( 25 )\end{matrix}$$

θ₁ is the angle when a continuous planar wave corresponding to the p\hat{ } (kₓ, k_{y}, ω) is incident on the holding member 140, and θ_{c} is a threshold angle of θ₁.

Equation (25) has the following meaning. The angle of incidence θ₁ on the holding member 140 is acquired using Equation (9) from the wave number of the spatial wave number component p\hat{ } (kₓ, k_{y}, ω). When the angle of incidence θ₁ is smaller than the threshold angle θ_{c}, Equation (24) is executed and correction is performed assuming that a longitudinal wave propagates through the holding member 140 (that is, c₂=c_{2L}). When the angle of incidence θ₁ is equal to or larger than the threshold angle θ_{c}, Equation (24) is executed and correction is performed assuming that a transverse wave propagates through the holding member 140 (that is, c₂=c_{2T}).

In the present embodiment, it is possible to reduce the computation amount by using a phase correction amount (Equation (25)) which can be calculated simply as compared to Equation (4) or (14). Furthermore, it is possible to correct the phase of a component which has been transformed into a transverse wave by switching the velocity in the holding member 140 to be used for correction between longitudinal waves and transverse waves according to the angle of incidence θ₁.

Equation (26) which is a modification of Equation (25) may be used.

$$\begin{matrix}[ Math . 14 ] & \\ {\hat{p}}_{cL} ( {k}_{x} , {k}_{y} , ω ) = \hat{p} ( {k}_{x} , {k}_{y} , ω ) \cdot exp ( i \vert k \vert T ( cos {θ}_{1} - \frac{{c}_{1}}{{c}_{2}} cos {θ}_{2} ) ) \\ {\hat{p}}_{cT} ( {k}_{x} , {k}_{y} , ω ) = \hat{p} ( {k}_{x} , {k}_{y} , ω ) \cdot exp ( i \vert k \vert T ( cos {θ}_{1} - \frac{{c}_{1}}{{c}_{2}} cos {θ}_{2} ) ) \\ {\hat{p}}_{c} ( {k}_{x} , {k}_{y} , ω ) = \{ \begin{matrix}{\hat{p}}_{cL} ( {k}_{x} , {k}_{y} , ω ) + {\hat{p}}_{cT} ( {k}_{x} , {k}_{y} , ω ) , & {θ}_{1} < {θ}_{c} \\ {\hat{p}}_{cT} ( {k}_{x} , {k}_{y} , ω ) , & {θ}_{1} \geq {θ}_{c}\end{matrix} & ( 26 )\end{matrix}$$

That is, when θ₁<θ_{c}, the spatial wave number component is corrected using both the longitudinal wave velocity c_{2L} and the transverse wave velocity c_{2T} and the sum thereof is obtained as a corrected spatial wave number component p_{c}\hat{ }. When θ₁≤θ_{c}, the spatial wave number component is corrected using the transverse wave velocity c_{2T} to obtain the corrected spatial wave number component pc\hat{ }. This reflects that, when the holding member 140 is thick, transverse and longitudinal waves propagate through the holding member 140 if the angle of incidence is smaller than the threshold angle and transverse waves only propagate if the angle of incidence is equal to or larger than the angle of incidence. By correcting transverse waves when the angle of incidence is smaller than the threshold angle, it is possible to increase the information amount and to improve the accuracy of the subject information. Although Equation (26) computes the correction amount of the longitudinal and transverse waves two times, since the computation amount is smaller than that of Equation (4) or (14), it is possible to calculate the correction amount at a high speed.

In Equation (26), when summing the corrected spatial wave number component corrected using the longitudinal wave velocity c_{2L} and the corrected spatial wave number component corrected using the transverse wave velocity c_{2T}, the weights applied thereto may be changed. That is, Equation (27) is executed.

$$\begin{matrix} [ Math . 15 ] & \\ {\hat{p}}_{c} ( {k}_{x} , {k}_{y} , ω ) = \{ \begin{matrix}W \cdot {\hat{p}}_{cL} ( {k}_{x} , {k}_{y} , ω ) + ( 1 - W ) \cdot {\hat{p}}_{cT} ( {k}_{x} , {k}_{y} , ω ) , & {θ}_{1} < {θ}_{c} , 0 \leq W \leq 1 \\ {\hat{p}}_{cT} ( {k}_{x} , {k}_{y} , ω ) , & {θ}_{1} \geq {θ}_{c}\end{matrix} & ( 27 )\end{matrix}$$

The weight W may be designated by an operator. According to an example of a designation method, the value W may be allocated to the slider 161 in FIG. 6 and the subject information may be displayed on the display unit 160 according to the value W changed by an operator. In this way, it is possible to display information more suitable for use in diagnosis or the like conducted by an operator. Moreover, during designation, the specification acquiring unit may also serve as the slider 161. The slider 161 corresponds to a correction characteristics adjusting unit of the present invention.

A table that stores the value of the weight W for each spatial wave number of the spatial wave number component may be stored in the processing unit 950 and the table may be used in Equation (27). By setting an optimal weight W based on longitudinal and transverse wave phase characteristics which depend on the angle of incidence θ₁ and ω in advance, it is possible to improve the contrast and the like of the subject information.

According to the subject information acquiring method according to the present embodiment, it is possible to acquire subject information with high accuracy (image quality) in which defocusing or the like is suppressed at a high speed.

The third embodiment may be executed immediately after the subject is measured, and subsequently, the second embodiment may be executed. According to this method, it is possible to check the subject information at an early stage by taking advantage of the speediness of the third embodiment and to immediately confirm that measurement was performed correctly. Moreover, by acquiring the high-accuracy subject information in which amplitude and phase are corrected according to the second embodiment, it is possible to further enhance the accuracy of diagnosis or the like.

A planar holding unit has been described in the respective embodiments. However, the present invention can be applied to a holding unit having an arbitrary shape. That is, the present invention can be applied by converting modulation of the spatial wave number component to modulation of the continuous planar wave according to the shape. An example of the shape of the holding member includes a bowl shape and a cup shape which follows the shape of the breast.

As described above, according to the present invention, it is possible to provide images with a small computation time while improving the image quality of photoacoustic imaging or ultrasound echo imaging. Therefore, when specific information is acquired on the basis of an acoustic wave propagating from a subject, it is possible to acquire high-accuracy information in a few seconds.

The present invention has been described in detail with reference to specific embodiments. However, the present invention is not limited to the specific embodiments and the embodiments may be modified without departing from the technical scope and spirit of the present invention.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-125805, filed on Jun. 24, 2016, which is hereby incorporated by reference herein in its entirety.

1. An apparatus that acquires specific information on a subject on the basis of a signal originating from an acoustic wave which has propagated from the subject and propagated through an acoustic medium disposed between the subject and a receiving unit,
the apparatus comprising:
a wave number acquiring unit configured to acquire a spatial wave number component of the signal;
a correcting unit configured to correct the spatial wave number component using parameters of a longitudinal wave velocity in the subject and a transverse wave velocity in the acoustic medium; and
an information acquiring unit configured to acquire the specific information using the corrected spatial wave number component.

2. The apparatus according to claim 1, wherein
the correcting unit is configured to correct the spatial wave number component using parameters of the longitudinal wave velocity in the subject, and longitudinal and transverse wave velocities in the acoustic medium.

3. The apparatus according to claim 1, wherein
the correcting unit is configured to correct at least one of a phase and an amplitude of the spatial wave number component.

4. The apparatus according to claim 3, wherein
the correcting unit is configured to perform correction using a complex amplitude transmittance, based on the parameter of the transverse wave velocity in the acoustic medium.

5. The apparatus according to claim 4, wherein
the correcting unit is configured to perform the correction by performing deconvolution on the spatial wave number component using the complex amplitude transmittance.

6. The apparatus according to claim 5, further comprising:
a correction characteristics adjusting unit configured to adjust correction characteristics of the deconvolution.

7. The apparatus according to claim 4, wherein
the complex amplitude transmittance is a transmittance set according to interference inside the acoustic medium.

8. The apparatus according to claim 4, wherein
the complex amplitude transmittance is a transmittance set regardless of interference inside the acoustic medium.

9. The apparatus according to claim 3, wherein
the correcting unit is configured to perform correction on phase modulation including transform of the acoustic wave in the acoustic medium into a transverse wave.

10. The apparatus according to claim 9, wherein
the correcting unit is configured to:
correct, using the parameters of the longitudinal wave velocity in the subject and the longitudinal and transverse wave velocities in the acoustic medium, the spatial wave number component, and,
implement switching between the correction using the longitudinal wave velocity in the acoustic medium and the correction using the transverse wave velocity in the acoustic medium, according to an angle of incidence on the acoustic medium, of a continuous planar wave corresponding to the spatial wave number component.

11. The apparatus according to claim 10, wherein
the correcting unit is configured to change a weight between the correction using the longitudinal wave velocity in the acoustic medium and the correction using the transverse wave velocity in the acoustic medium.

12. The apparatus according to claim 11, further comprising:
a correction characteristics adjusting unit configured to enable the weight to be adjusted.

13. The apparatus according to claim 1, further comprising:
a relating information acquiring unit configured to acquire relating information on the acoustic medium, wherein
the correcting unit is configured to acquire the parameters using the relating information.

14. The apparatus according to claim 1, wherein
the acoustic medium is a holding unit for holding the subject.

15. The apparatus according to claim 1, further comprising:
the receiving unit configured to output the signal by receiving the acoustic wave.

16. (canceled)

17. An information processing method of acquiring specific information on a subject on the basis of a signal originating from an acoustic wave which has propagated from the subject and propagated through an acoustic medium disposed between the subject and a receiving unit,
the method comprising:
a wave number acquisition step of acquiring a spatial wave number component of the signal;
a correction step of correcting the spatial wave number component using parameters of a longitudinal wave velocity in the subject and a transverse wave velocity in the acoustic medium; and
an information acquisition step of acquiring the specific information using the corrected spatial wave number component.

18. A non-transitory storage medium storing a program for causing a computer to execute the information processing method according to claim 17.
